SEQUENCE LISTING (20 sequences)

SEQ ID NO: 1 - DNA, 22 bp, ARTIFICIAL
  SYNTHETIC, PCR PRIMER
aagaagctgagccgggttgatg

SEQ ID NO: 2 - DNA, 31 bp, ARTIFICIAL
  SYNTHETIC, PCR PRIMER
ggcggaattcaacctcgtatcccgtcagcgt

SEQ ID NO: 3 - DNA, 30 bp, ARTIFICIAL
  SYNTHETIC, PCR PRIMER
gtagaaagcttcctctcactacccaagacc

SEQ ID NO: 4 - DNA, 22 bp, ARTIFICIAL
  SYNTHETIC, PCR PRIMER
cttgttctcaacgccatccagc

SEQ ID NO: 5 - DNA, 21 bp, ARTIFICIAL
  SYNTHETIC, PCR PRIMER
agcactccttcctccctcgtg

SEQ ID NO: 6 - DNA, 22 bp, ARTIFICIAL
  SYNTHETIC, PCR PRIMER
tcctatacttgccactcagccc

SEQ ID NO: 7 - DNA, 22 bp, ARTIFICIAL
  SYNTHETIC, PCR PRIMER
gcggattaccaacactaccacc

SEQ ID NO: 8 - DNA, 21 bp, ARTIFICIAL
  SYNTHETIC, PCR PRIMER
acaccgtctccgtctgccttc

SEQ ID NO: 9 - DNA, 22 bp, ARTIFICIAL
  SYNTHETIC,PCR PRIMER
agccatgacctgagtcagtcag

SEQ ID NO: 10 - DNA, 21 bp, ARTIFICIAL
  SYNTHETIC,PCR PRIMER
atgccgaacttcctctgcgcc

SEQ ID NO: 11 - DNA, 23 bp, ARTIFICIAL
  SYNTHETIC, PCR PRIMER
ttgagcttctttctatcagggtc

SEQ ID NO: 12 - DNA, 22 bp, ARTIFICIAL
  SYNTHETIC, PCR PRIMER
ttcgactttgtccattgccgcc

SEQ ID NO: 13 - DNA, 21 bp, ARTIFICIAL
  SYNTHETIC, PCR PRIMER
tgaatggccggagagtgcacg

SEQ ID NO: 14 - DNA, 21 bp, ARTIFICIAL
  SYNTHETIC, PCR PRIMER
cctggtacgttgcctttgtag

SEQ ID NO: 15 - DNA, 9759 bp, ASPERGILLUS NIDULANS
  misc_feature (1)..(9759): tdi gene cluster
ctacaggccccgctccctcagtacattcttcaatgtccctgcaaaagcctcaacatactcccgattcaacatggtgtaatgtgctccttgcacatcgtggaaacgcacgtcctcccgcacaaactccttccaggcacttaacctcccctccacccagtcgattcggtcctttgcaacatggctcaaagggtccgccacaaacacatccatacacttgactgatcccgagggttcatagtccaccgccagactctgcatattagatgctagacctacccagagaagatagtactcctctgaaaggccgagctcgtcccaccgtgcggcatcgcagtgctggcgcaggtaccggatggcgtccagacggcggtttgcccgattaaactcctgcagagtaggtttatgcgtatacgctgccagctcggtcatcagaccgacgaagtagaatagatggatcacgcactcctcccagacgagctcacgcatgcggaacttgatatgcggcggaagattccagctcccgcagtagcggacctcatcgccgtcctgttcgagaagcttgctgacttcaaatgccaccatcccgccaaacgagtaccccgctattgcgtagggcccgtggggctggcgttctttaattgcgtcgcggtaggttgtgaacaattcctccagtgaggtgaagggggtttcgggcaagccggccgccgcgttgaagccttttgcgcggaaggcgtaaacgggtcggtctgtaatgtgatgggccaggttcacgaagactaggacctcaccgacgcccgggtgtaccagccatagaggactcttggtcccgtgcggttgcagggtcacgacggggtcgtagacgtgcgtggaagactgatcctgtgagcgaggcgcggcccctgttgcgagggcgacggctagccccctggctgtcgagtctttgaggatgtctgttaggcggaggggctgagaaggctgtaggcacttattgatgcggtggatgatagcgactagatccatcgacgtggcgcctattgagaggattgaatcgttgacgccaaagctgtcatcatcagaccggatctccagctgttccttgataatatccagaattactgcttcatccggcgtctctgggctcgcacgggtcttttgctggtagcgtcttatagcctcgtcgttgatctgctgctgcgtagcgaactggccttcttccagagccgtcttcagttttgcgcgcgacagttttcccagcgtgctctttggcatatcctgcggacgcagcggcactacgcgcggccgggaccgcgtgtgcatggccacgacacggatgatgctgctttgcgtgctgaacctggcttcgtcatcgctctccacataagatggaaggtagagcacaaccacgacctcggtatccatggttgcatcgcggctgctgaacgtgcagaagtaactaggtgttgcgcctgggatctgcgcctgctcgagagcagcatccagttcgtacgggaggtatttgactccattgatgttgatcatctccttcgtgcgcccgtcgaggtgcagattgccgttgctgtcaatgaacgccagatcccccgtccggaaccatccatcgctggtgaacgcctctgctgtggcggcaggattattgtagtaacctttaaagacaacttccccggttacttcgaggctgccgcgctcaccgggggctgcctcttcgctcggagtgtcaagccgtgtcacccgcattcgcactccaggcatcggtttcccgagacaggcgaactcatggcgctgggcgtgatcatagcttgggcagtgcgagttgaagatacatccggccacggtttcggtcataccgaaggagggcttgaaaacgttgtcgggagccccgtaccggctgaggagggattggagtgcaatacaaacctctgtgacgttcgcctcaccaccggtatcaatatagagcgtctcaaggttgaggccggggtccaggatatactctggactccccgactccagctgtcgccgcaacttggcgcagaggaagttcggcatgaacgtgcgcgagacgcggtgtctgcttatcaggttaagaagctgagccgggttgatgagaagatccggagcagggacttgaatctgtgatatgccggacacgatggcgaagatatggcagtggactagattggcgacgtggtccatgtgcacccaggagaggaacgggctgcgggggaagcggaggctggccgcggtggacttgcccctgaaggccgcaaggagctgttgatgggtcagagggacagcttttgcgttgccgctgctcccggaggtcagcatgagggcaagcatatcggtcgaagacggggttagggcaggcagaggtgcgtcagcaacgtccgcaatttcgggagctgcgaggatctcatccactgttcgagctttgatccggtcatccgctgtctgctcttcaaagggggccaagagggcaggccgggtcagacagaccggtgaattgagcgtctcggacagatgacgcagatgcctctctctatctgccgggttctggctgaacatcccaggcccggtgagggcaggtatgcccccagccagaaggacagaccagtaccatacgatgctgtccagtgcggactcaaagtgaacgaggacaatggacttggggctacatagcttctgctgcaacagtctggtggcattcgcctctgcctgatgcagcagatctttgtaggagactgtctgtggaggtgatgaggtgctgatgctgtttgggtggtatactataatgccctcatcggtatgagcagcagcatgtcgaagagcgtccacgatgttgccaaacgggtacttggctgccctgagcggtgcgatctcggtcttgcttggtgccatcttgttacagtctaagaggaggtcctagcctggccagaagggtctcaatgagtgagttatgagtaagttgggtgagccactgtgcctgtttctccgcactcaagacactttaagtatgcagcctgccctaatacgagatattcccgtcctcgcggggtaagtccaaatcaggcccgtgttgcacaatgacgatactattattattactcgttcatattacacgctgacgggatacgaggttgcattccgccacacgagataccaattcaaggtcacaaaaggacaagctgcagccgggcctggaccatggggcgtatatatgatgacagtagagtactctgaaattccttgcaaacagtcctttcttttgcgcagaaactgtcttcatcatggctacagaatactggtcccgtcatctacgctcagtgctggctccgctgttcgctgcagctggcacatactctcctgaagatcaggagtcccatctggccttcattgacgagcacattgcgcccaacctgggccctctcccttgggagccccatggaccctacagcactccttcctccctcgtgggctcccccttcgaccccagcatcaacatcgtctcatccggaaaggccaaggtccgtttcgactttgacgtgatcagtccacctgatcgaacaggcccagacccctttgcagagggatccgccagggagatcctccaccgtctcgccgaccttgtcggcgcagacacacagtggatgggctacctcatggatgctctctacctgacccccgcggaggctgaggttgcgaaaacgaagttgcctccaggtgttgctatcccgcccagctcagtgggcttcgacttcgacggccccgagcggacgctgaagttctacatccccagtgtgcggaaagcgctagcaacggggcaggatgtgtccgaactcatgctaaagactctacgtggattacagccacttgggtctgagctagtgccagcgatggacctgattgcttcgtaagctctaacttcacatctctagattattctgtattagcgcgatgcaagctgactgaaccaggtatctctcaacccgcaccaacgacgccatgctcccgctcgtcgggattgactgcctcgatccaaggacacataagaacgctcgggtcaaatgctacctgcacacgagcagcaacagcttcgcagtcgtccgcgacgtcctcacgctgggaggccgtctcagcgacgacacctcgctcaagcgagtcgagacactcaaatcggtctggcctttgctgatcaacgagctagaaggtccacagagcgacgcggccaccatggacgaatcctggtccaagccagagcggctcaaccggacagggtactcggggatccagtacacgattgagattacccccggtcaggcaatccccgacacgaagatctacgtcccgttattccagtacacggacagctctgaggtcgctgagcggaactttgagagcgcgctaaagaagctggggaatgagtgggggctgagtggcaagtataggagcgtcatgcaggaaatcttgtgagtcatttcttctatttcatttatctattttttattgttatacgatcccttctgacggccgcaaagcaaggatgtagagaactatggccagacgtacgcatctttctcctacacggagggaaagggcgtctatacgacctcgtatgttgctatgcctattaaggatgagggagggggtagcctcgctggagatttcggtttcaggaactagatcaagtactcggtgctgtagcgctcaatcagatactagtctaatcaaatcattggctgtcgttgattcggagtgtaggaactatttcttagtagacatcttcctctcactacccaagacctccgccaaaacctagcacccaaatgagcgcggagcatccggctgtcccccacattgacatcagatgccaagcatcgagcaaagtaaagaaaggtctcgaccagatctctgcctcattccttttgagtgggatttcgtcagctgcatgcccagaaagctagggctgcttagtacgggacgggccagctgcacctgcagcttggcttcgtatcccagaatagccaacatttatccggagactcgcacattgtatccccttgtggcgattcgagattttgagcttctttctatcagggtctaattcttactgcataatgcggggttgattgtcgtacgtactaaggtcaacatgcacacggcctacctgcaacctttcagcctacaagatgtaacccaagaaaacccagagtataaaaaagcgcaaataatataggctgacctcgactccatctcattcttcagcctaccatcttgctatttaggttaactccgcatcgcagagaaacgactcacccagaacggaaacgactatgcatgccgctctcgtcccaacctggtcctccccctgtcccatctataccgagattccagacccggggcctccgccaccagaacagctccaactgaaagtcttggccgttgggattccccgcgttgtacggctccgtgctcgagggatccacccgactgccaagtcagccagtctcccgtacgaccccagcattgacggcgtagggattgacgaacaaaccggcattatgtactacattcttccgctctcggcgtcttgtcttgcggagaaagtgaatgtggaccgggacaatctggtcccgctccagcccggggcacctaaacctcaacctcgaaatggacctgaaaatggatatggaattgcacttggcgatgctgctgaccatagagctgagacgctggaccccatcgcaattgcggggctcgtgaatcccgtgtcgagtagctggatggcgttgagaacaagggtggatggcgaaattacggggaaaacggtcctggttctgggggcaacgagtaagagtgggcgagccgcagtgctcgttgcaaggtttctgggagcaaataaagtgattggagtggcgaggagagaggaagggctaagaagtgtggaagggttggatggctgggttacctcgggggatatgcttcctggggagaccggggttaggtttgctctaccggactgggttgggccagtgcatatcgtcctggattacgttggaggcagcgttgccgcgggagtcctgggcagtgctgagattgaagagggtagagagttgcaatacgtgcaggttgggaacctcgcgcttgagctgggcacgggagagaagcatatgtttgaaaccttaccaggccacctgatcagccggaagcccatctgcatccgcggatcagggatgggaagtttcagtaggagggatctggtccgagagatgcctggcttggtggcatttcttgcacgaatgaaggcgccgtttgggattgcgagcgcgccgatgtgcgaggtggcgtcggtttggcaggatgaggatacgaagggaagccgagtggtgattgtaccttgaacgacacttgtacgaactggggcggtttgtcatgacacattcaacacggactgacagttacaatcatggtagtagtagcctgagatcccgaatacgaagccattcctgtggttcatcgccttctatgctatatagaggcttttaattttgctggtgtctagtatcaaacccactactgtagaccatcgtgctactgaatatcgtaccaacatcaccatgggctcaataggggccaataatgctgtggcggacccaactcctcttttctcgtcacgggtgcagaaatgggagcccggtgcaagtaagccacgttggccatgacagcgcaattccaagctgaccagatcttcagtccggagcctactccctctcgaagcccttcctggcatgatctccctcgtggcaggaaaacccagtcccgagacgttccccattgctgagatcgccatctcactcaaggatactccagccgggacaggtaggatcgtggttgatggggacgagctcaaccaggctcttcaatatggtcttcctcgaggaaatgcccagctaatccaggtttgctctgtcccttagtctccatatttcctactgatcattgttgttcagtggttcgaaagtcttcagaggagcgtccacggcctcgatgagaacggaggctggtcgtgctgcattggcacatggaagtcaggagctcattcatcgcgtcatccaggtcttcactgatcctggggacccagttcttctcgaaacgtgggtctcactccctttgtatgcattcccggacgcaggcgccgctgactgaagccatagaccagcatatccgtatgatttcctccaggaattttcagagtggacattttagcaccttgctgacagggaccacagaggcgtcgccgggttccttcgcgcagacggacaggagcttatcccggtctactctgacgcccagggcctcaatccggccagcctggagcaggcattgtcggagtggcccggagactcgccgcgccccaaagtcctctatacaaccccgactgggtcgaacccaaccggtcaatcctgcaccgagagccgcaaggccgagatgtaattactcctctgcccagccactgctccagacctgagactgacagttccatcccgacacctatagtctccgtctggcaaagcgattcaattttatcatcctcgaagacgacgcatactattatctcaactacggagacgacaaacagagagcgcggagctatctcgccctcgaaagagacgtcaatggagagtcaggccgcgtcgtgcgtttcgactcgctcagcaagatcgtcagtcccggcatgcgtctggggatcctcacagcacaagcagcggttgttgacaaggtggtgcggatcactgaaaatataaagtatgtttatccttctctcttctcctgtcctgttctggctgcttcaaatgactcgctcactgaatcccagccttcaaccctcctccacaactcaacttctagccctctctctactccgccactggggtcaagctggcttcctgaaacactgtgcagaagccgccgaggtctatcgacgtcgccgagacgtctttgtttctgcggcggagcggcatctccagggtcgagctacatgggtggttccaacggccggcatgtttgtctggctggaacttaagttgccaccggaaatggatagcttcgagcttctgaagagccagggaatgaagaatggggttcttgctattcctggtgttgccttcatgcctgggaacgagcaaacgtgctatatccgggtgagcttcagtctagttcctgagagagatatggatgaggcgtgtaggcggatcgcgggtctagttgatcgatgtgcttgccattcctaagcttattatacttttgttcgatggttctaatactattttcccggtcaaaagatcttcgccatgaatttggttcactcgctatctgtaatcacgtcggatccaggtgcagctggcttctgcccatacacaacatatctattcgctcgactcagcatataagacctcgtaggacagtcatgaaaagggatggtagacttacaccggccagtaaccatgcacaccctcattcatcagctcctcctttacgttccccaagaacgtctttgcatcatccatctcccatccaagtacttttaccaacaaagccaccgaaaactcttcaaagcgctgcaacacattgtacagattccaaagacccaaggtcttacccttttcatccgcaggccacgggttcgtgggccaattatacgcaacggtgtggcaattcacgaatccggcctcgcgcatccacgtctcatatttggcaggattgtccacgggacggttcatcttctttgaggcctctataagaagacggccccattgggagagcgggttgttctctgatagggttccgtcgtcgcttgtgacggggttactgagttcttgcatctccagccagccgccgggagtgagggagctttttgccagtgagaccgggctcagcacgattaagcgaatggtacttgcctgaacgcctgcttgaagagtcggggctcgtctaggcgccggtgatgctggcggcaatggacaaagtcgaactggcgggtgtatgtccatggttcgttggcgtcgcttgcgtctggaataacactgagacctagaggtattttacttgtgggactaggatcgttggccacgatatacgcagaaggattctgttcagctgctaccggttaggggatatctagtgtatatagaccgagaatgctgcagcctaccaaaagcatggacccatgttgagtctcctgttgctatatcgagcacagattgcactggtgacggtaggggagataactgaagtttcccatctagggttagatggaagagctggtgctggagtgcttggtggtgtcagtctggtacgccttgaagtttctatgcaaccactctatcttatccacgtagacggacgatggactaagcttaccaagtcgctctgtctgtgagacaggtttcgcgtcagcattgcgttcatagtcagggatcataaacactgcctgatacggacctcgatatcccgttctgtaaggggctcaattagcttagactggtaacgtatacatctaaatcttcatacctccattattggttttctgaaacagtccattcctcgcttgtccactgttgtccaggtacttttgtacggactcggcgaccgcattggcaaggtcgttcctgggtcagttagatattgctgtaaaatatgtctactagtgtcaaggacgtacatgtttgcgtacgggcggttaaccat

SEQ ID NO: 16 - DNA, 2913 bp, ASPERGILLUS NIDULANS
  cDNA tdiA of Aspergillus nidulans
atggcaccaagcaagaccgagatcgcaccgctcagggcagccaagtacccgtttggcaacatcgtggacgctcttcgacatgctgctgctcataccgatgagggcattatagtataccacccaaacagcatcagcacctcatcacctccacagacagtctcctacaaagatctgctgcatcaggcagaggcgaatgccaccagactgttgcagcagaagctatgtagccccaagtccattgtcctcgttcactttgagtccgcactggacagcatcgtatggtactggtctgtccttctggctgggggcatacctgccctcaccgggcctgggatgttcagccagaacccggcagatagagagaggcatctgcgtcatctgtccgagacgctcaattcaccggtctgtctgacccggcctgccctcttggccccctttgaagagcagacagcggatgaccggatcaaagctcgaacagtggatgagatcctcgcagctcccgaaattgcggacgttgctgacgcacctctgcctgccctaaccccgtcttcgaccgatatgcttgccctcatgctgacctccgggagcagcggcaacgcaaaagctgtccctctgacccatcaacagctccttgcggccttcaggggcaagtccaccgcggccagcctccgcttcccccgcagcccgttcctctcctgggtgcacatggaccacgtcgccaatctagtccactgccatatcttcgccatcgtgtccggcatatcacagattcaagtccctgctccggatcttctcatcaacccggctcagcttcttaacctgataagcagacaccgcgtctcgcgcacgttcatgccgaacttcctctgcgccaagttgcggcgacagctggagtcggggagtccagagtatatcctggaccccggcctcaaccttgagacgctctatattgataccggtggtgaggcgaacgtcacagaggtttgtattgcactccaatccctcctcagccggtacggggctcccgacaacgttttcaagccctccttcggtatgaccgaaaccgtggccggatgtatcttcaactcgcactgcccaagctatgatcacgcccagcgccatgagttcgcctgtctcgggaaaccgatgcctggagtgcgaatgcgggtgacacggcttgacactccgagcgaagaggcagcccccggtgagcgcggcagcctcgaagtaaccggggaagttgtctttaaaggttactacaataatcctgccgccacagcagaggcgttcaccagcgatggatggttccggacgggggatctggcgttcattgacagcaacggcaatctgcacctcgacgggcgcacgaaggagatgatcaacatcaatggagtcaaatacctcccgtacgaactggatgctgctctcgagcaggcgcagatcccaggcgcaacacctagttacttctgcacgttcagcagccgcgatgcaaccatggataccgaggtcgtggttgtgctctaccttccatcttatgtggagagcgatgacgaagccaggttcagcacgcaaagcagcatcatccgtgtcgtggccatgcacacgcggtcccggccgcgcgtagtgccgctgcgtccgcaggatatgccaaagagcacgctgggaaaactgtcgcgcgcaaaactgaagacggctctggaagaaggccagttcgctacgcagcagcagatcaacgacgaggctataagacgctaccagcaaaagacccgtgcgagcccagagacgccggatgaagcagtaattctggatattatcaaggaacagctggagatccggtctgatgatgacagctttggcgtcaacgattcaatcctctcaataggcgccacgtcgatggatctagtcgctatcatccaccgcatcaataagtgcctacagccttctcagcccctccgcctaacagacatcctcaaagactcgacagccagggggctagccgtcgccctcgcaacaggggccgcgcctcgctcacaggatcagtcttccacgcacgtctacgaccccgtcgtgaccctgcaaccgcacgggaccaagagtcctctatggctggtacacccgggcgtcggtgaggtcctagtcttcgtgaacctggcccatcacattacagaccgacccgtttacgccttccgcgcaaaaggcttcaacgcggcggccggcttgcccgaaacccccttcacctcactggaggaattgttcacaacctaccgcgacgcaattaaagaacgccagccccacgggccctacgcaatagcggggtactcgtttggcgggatggtggcatttgaagtcagcaagcttctcgaacaggacggcgatgaggtccgctactgcgggagctggaatcttccgccgcatatcaagttccgcatgcgtgagctcgtctgggaggagtgcgtgatccatctattctacttcgtcggtctgatgaccgagctggcagcgtatacgcataaacctactctgcaggagtttaatcgggcaaaccgccgtctggacgccatccggtacctgcgccagcactgcgatgccgcacggtgggacgagctcggcctttcagaggagtactatcttctctgggtaggtctagcatctaatatgcagagtctggcggtggactatgaaccctcgggatcagtcaagtgtatggatgtgtttgtggcggaccctttgagccatgttgcaaaggaccgaatcgactgggtggaggggaggttaagtgcctggaaggagtttgtgcgggaggacgtgcgtttccacgatgtgcaaggagcacattacaccatgttgaatcgggagtatgttgaggcttttgcagggacattgaagaatgtactgagggagcggggcctgtag

SEQ ID NO: 17 - DNA, 1260 bp, ASPERGILLUS NIDULANS
  cDNA tdiB of Aspergillus nidulans
atggctacagaatactggtcccgtcatctacgctcagtgctggctccgctgttcgctgcagctggcacatactctcctgaagatcaggagtcccatctggccttcattgacgagcacattgcgcccaacctgggccctctcccttgggagccccatggaccctacagcactccttcctccctcgtgggctcccccttcgaccccagcatcaacatcgtctcatccggaaaggccaaggtccgtttcgactttgacgtgatcagtccacctgatcgaacaggcccagacccctttgcagagggatccgccagggagatcctccaccgtctcgccgaccttgtcggcgcagacacacagtggatgggctacctcatggatgctctctacctgacccccgcggaggctgaggttgcgaaaacgaagttgcctccaggtgttgctatcccgcccagctcagtgggcttcgacttcgacggccccgagcggacgctgaagttctacatccccagtgtgcggaaagcgctagcaacggggcaggatgtgtccgaactcatgctaaagactctacgtggattacagccacttgggtctgagctagtgccagcgatggacctgattgcttcgtatctctcaacccgcaccaacgacgccatgctcccgctcgtcgggattgactgcctcgatccaaggacacataagaacgctcgggtcaaatgctacctgcacacgagcagcaacagcttcgcagtcgtccgcgacgtcctcacgctgggaggccgtctcagcgacgacacctcgctcaagcgagtcgagacactcaaatcggtctggcctttgctgatcaacgagctagaaggtccacagagcgacgcggccaccatggacgaatcctggtccaagccagagcggctcaaccggacagggtactcggggatccagtacacgattgagattacccccggtcaggcaatccccgacacgaagatctacgtcccgttattccagtacacggacagctctgaggtcgctgagcggaactttgagagcgcgctaaagaagctggggaatgagtgggggctgagtggcaagtataggagcgtcatgcaggaaatcttcaaggatgtagagaactatggccagacgtacgcatctttctcctacacggagggaaagggcgtctatacgacctcgtatgttgctatgcctattaaggatgagggagggggtagcctcgctggagatttcggtttcaggaactag

SEQ ID NO: 18 - DNA, 1068 bp, ASPERGILLUS NIDULANS
  cDNA tdiC of Aspergillus nidulans
atgcatgccgctctcgtcccaacctggtcctccccctgtcccatctataccgagattccagacccggggcctccgccaccagaacagctccaactgaaagtcttggccgttgggattccccgcgttgtacggctccgtgctcgagggatccacccgactgccaagtcagccagtctcccgtacgaccccagcattgacggcgtagggattgacgaacaaaccggcattatgtactacattcttccgctctcggcgtcttgtcttgcggagaaagtgaatgtggaccgggacaatctggtcccgctccagcccggggcacctaaacctcaacctcgaaatggacctgaaaatggatatggaattgcacttggcgatgctgctgaccatagagctgagacgctggaccccatcgcaattgcggggctcgtgaatcccgtgtcgagtagctggatggcgttgagaacaagggtggatggcgaaattacggggaaaacggtcctggttctgggggcaacgagtaagagtgggcgagccgcagtgctcgttgcaaggtttctgggagcaaataaagtgattggagtggcgaggagagaggaagggctaagaagtgtggaagggttggatggctgggttacctcgggggatatgcttcctggggagaccggggttaggtttgctctaccggactgggttgggccagtgcatatcgtcctggattacgttggaggcagcgttgccgcgggagtcctgggcagtgctgagattgaagagggtagagagttgcaatacgtgcaggttgggaacctcgcgcttgagctgggcacgggagagaagcatatgtttgaaaccttaccaggccacctgatcagccggaagcccatctgcatccgcggatcagggatgggaagtttcagtaggagggatctggtccgagagatgcctggcttggtggcatttcttgcacgaatgaaggcgccgtttgggattgcgagcgcgccgatgtgcgaggtggcgtcggtttggcaggatgaggatacgaagggaagccgagtggtgattgtaccttga

SEQ ID NO: 19 - DNA, 1311 bp, ASPERGILLUS NIDULANS
  cDNA tdiD of Aspergillus nidulans
atgggctcaataggggccaataatgctgtggcggacccaactcctcttttctcgtcacgggtgcagaaatgggagcccggtgcaatccggagcctactccctctcgaagcccttcctggcatgatctccctcgtggcaggaaaacccagtcccgagacgttccccattgctgagatcgccatctcactcaaggatactccagccgggacaggtaggatcgtggttgatggggacgagctcaaccaggctcttcaatatggtcttcctcgaggaaatgcccagctaatccagtggttcgaaagtcttcagaggagcgtccacggcctcgatgagaacggaggctggtcgtgctgcattggcaatggaagtcaggagctcattcatcgcgtcatccaggtcttcactgatcctggggacccagttcttctcgaaacaccagcatatccaggcgtcgccgggttccttcgcgcagacggacaggagcttatcccggtctactctgacgcccagggcctcaatccggccagcctggagcaggcattgtcggagtggcccggagactcgccgcgccccaaagtcctctatacaaccccgactgggtcgaacccaaccggtcaatcctgcaccgagagccgcaaggccgagattctccgtctggcaaagcgattcaattttatcatcctcgaagacgacgcatactattatctcaactacggagacgacaaacagagagcgcggagctatctcgccctcgaaagagacgtcaatggagagtcaggccgcgtcgtgcgtttcgactcgctcagcaagatcgtcagtcccggcatgcgtctggggatcctcacagcacaagcagcggttgttgacaaggtggtgcggatcactgaaaatataaaccttcaaccctcctccacaactcaacttctagccctctctctactccgccactggggtcaagctggcttcctgaaacactgtgcagaagccgccgaggtctatcgacgtcgccgagacgtctttgtttctgcggcggagcggcatctccagggtcgagctacatgggtggttccaacggccggcatgtttgtctggctggaacttaagttgccaccggaaatggatagcttcgagcttctgaagagccagggaatgaagaatggggttcttgctattcctggtgttgccttcatgcctgggaacgagcaaacgtgctatatccgggtgagcttcagtctagttcctgagagagatatggatgaggcgtgtaggcggatcgcgggtctagttgatcgatgtgcttgccattcctaa

SEQ ID NO: 20 - DNA, 891 bp, ASPERGILLUS NIDULANS
  cDNA tdiE ofAspergillus nidulans
atggttaaccgcccgtacgcaaacatgaacgaccttgccaatgcggtcgccgagtccgtacaaaagtacctggacaacagtggacaagcgaggaatggactgtttcagaaaaccaataatggagaacgggatatcgagacagagcgacttgttatctcccctaccgtcaccagtgcaatctgtgctcgatatagcaacaggagactcaacatgggtccatgcttttggtctcagtgttattccagacgcaagcgacgccaacgaaccatggacatacacccgccagttcgactttgtccattgccgccagcatcaccggcgcctagacgagccccgactcttcaagcaggcgttcaggcaagtaccattcgcttaatcgtgctgagcccggtctcactggcaaaaagctccctcactcccggcggctggctggagatgcaagaactcagtaaccccgtcacaagcgacgacggaaccctatcagagaacaacccgctctcccaatggggccgtcttcttatagaggcctcaaagaagatgaaccgtcccgtggacaatcctgccaaatatgagacgtggatgcgcgaggccggattcgtgaattgccacaccgttgcgtataattggcccacgaacccgtggcctgcggatgaaaagggtaagaccttgggtctttggaatctgtacaatgtgttgcagcgctttgaagagttttcggtggctttgttggtaaaagtacttggatgggagatggatgatgcaaagacgttcttggggaacgtaaaggaggagctgatgaatgagggtgtgcatggttactggccggtatatgttgtgtatgggcagaagccagctgcacctggatccgacgtgattacagatagcgagtga

(19) United States
(12) Patent Application Publication
(10) Pub. No.: US 2009/0011476 A1
(43) Pub. Date: Jan. 8, 2009

(54) GENE CLUSTER AND METHOD FOR THE BIOSYNTHESIS OF TERREQUINONE A

(75) Inventors: Dirk HOFFMEISTER, Freiburg (DE); Nancy P. KELLER, Madison, WI (US)

Correspondence Address: QUARLES & BRADY LLP, 411 E. WISCONSIN AVENUE, SUITE 2040, MILWAUKEE, WI, 53202-4497, US

(73) Assignee: Wisconsin Alumni Research Foundation

(21) Appl. No.: 11/465,996

(22) Filed: Aug. 21, 2006

Related U.S. Application Data
(60) Provisional application No. 60/709,969, filed on Aug. 19, 2005.

(51) Int. Cl. C12P 17/10; C12N 15/11; C12N 1/20; C12N 1/19

(52) U.S. Cl. 435/121; 536/23.1; 435/252.3; 435/254.11; 435/254.2; 435/252.33

(57) ABSTRACT

The present invention provides a novel gene cluster containing five genes (tdiA-E) involved in indole alkaloid synthesis. Disruption of tdiB, encoding an enzyme with prenyltransferase activity, transferring dimethylallylpyrophosphate to C-2 of an indole structure, eliminated the production of the antitumor compound terrequinone A, a metabolite not known from A. nidulans. The invention further provides a method for expressing terrequinone A in a host cell and isolating purified terrequinone A therefrom.

### CROSS-REFERENCE TO RELATED APPLICATIONS

This application claims the benefit of U. S. Provisional application 60/709,969 filed Aug. 19, 2005 incorporated herein by reference.

### STATEMENT AS TO RIGHTS TO INVENTIONS MADE UNDER FEDERALLY SPONSORED RESEARCH OR DEVELOPMENT

This invention was made with United States government support awarded by the following agencies: NSF MCB-0196233, NSF MCB-0236393 and NIH F32 AI052654.

### FIELD OF THE INVENTION

This invention relates generally to the field of secondary metabolite production in fungi. In particular, this invention is directed to a gene cluster encoding the biosynthetic enzymes responsible for terrequinone A production in Aspergillus nidulans.

### BACKGROUND OF THE INVENTION

Filamentous fungi display many unique characteristics that render them of great interest to the research and medical communities. Among these characteristics is the biosynthesis of natural products which display a broad range of useful activities for pharmaceutical and agricultural purposes, e.g. antibiotic, immunosuppressant, lipid-lowering, or antifungal properties. Other attributes include the, so far, less desired potent phyto- and mycotoxic activities exhibited by fungal pathogens. These bioactivities of natural products have spurred efforts towards identifying genes involved in their biosynthesis. Accumulating data from studies of known secondary metabolite biosynthetic genes dispelled an original premise that fungal metabolic genes would be scattered throughout the genome. Instead, the hallmark of secondary metabolite genes—in contrast to genes involved in primary metabolism—is that they are clustered in fungal genomes. Examples of secondary metabolite gene clusters include those synthesizing pharmaceuticals of clinical use, such as the important β-lactam-antibiotics penicillin (PN) and cephalosporin, the antihypercholesterolaemic agent lovastatin and the ergopeptines with their important pharmacophore D-lysergic acid amide as well as carcinogenic toxins (aflatoxin and sterigmatocystin, ST).

Discovery of the first fungal gene clusters was largely a result of mutant searches followed by complementation with gene transformation. More sophisticated identification techniques arose when it was realized that many of the structural genes involved in secondary metabolism are highly conserved and could be cloned by hybridization probing or amplified from the fungal genome by use of degenerate primers; the latter technique was especially fruitful in procuring polyketide synthases (PKS). This conservation of DNA and protein sequences, coupled with the cluster motif of metabolic pathways, greatly facilitated the assignment of putative secondary metabolite genes in the completed Aspergillus genomes (Broad Institute and TIGR). Sequence alignments suggest A. nidulans has the potential to generate up to 27 polyketides, 14 non-ribosomal peptides, one terpene and two indole alkaloids; similar predictions can be made from the A. fumigatus and A. oryzae genomes. Interestingly, there appears to be almost no orthologs among these genes across the three species, thus representing a loss of synteny to a degree not seen in other regions of the genomes. This high number of putative metabolites is greater than the known metabolites ascribed to these species and may be a reflection of incomplete natural product analysis in these species and/or failure of many clusters to be expressed, at least under the culture conditions commonly used in laboratories. For example, the aflatoxin gene cluster is not transcribed in A. oryzae.

An intimation of a method to identify transcriptionally active clusters arose from the discovery of LaeA, a nuclear protein regulating secondary metabolite production in Aspergillus spp. Loss of LaeA silenced ST and PN production in A. nidulans and gliotoxin production in A. fumigatus whereas over expression of the gene increased PN and lovastatin production in A. nidulans and A. terreus, thus leading to the hypothesis that LaeA was involved in global regulation of secondary metabolite gene clusters in this genus (Bok, J. W. and Keller, N. P. (2004), LaeA, a regulator of secondary metabolism in Aspergillus spp. Eukaryot. Cell 3, 527-535). Such global regulation differs from the Streptomyces coelicolor A3 transcriptional regulator which is species specific.

Given the transcriptional nature of regulation by LaeA, the inventors considered this protein a promising gateway towards designing a novel genome-wide procedure to identify fungal natural products and described in U. S. Pat. No. 7,053,204 hereby incorporated in its entirety for all purposes. Therefore, the inventors predicted that transcribed secondary metabolite clusters would be revealed in a microarray profiling of laeA deletion (ΔlaeA) and over expression (OE::laeA) mutants allowing for the targeted manipulation and chemical characterization of novel natural products. This strategy and ultimately provided methods and techniques for the production and isolation of therapeutic and beneficial secondary metabolites.

### SUMMARY OF THE INVENTION

The present invention provides a novel gene cluster containing five genes (tdiA-E) involved in indole alkaloid synthesis. Disruption of tdiB, encoding an enzyme with prenyltransferase activity, transferring dimethylallylpyrophosphate to C-2 of an indole structure, eliminated the production of the antitumor compound terrequinone A, a metabolite not known from A. nidulans. The invention further provides a method for expressing terrequinone A in a host cell and isolating purified terrequinone A therefrom.

In one preferred embodiment, this invention provides an isolated terrequinone A gene cluster as set forth in SEQ ID NO: 15.

In another preferred embodiment, this invention provides an isolated gene from the terrequinone A gene cluster wherein the gene is selected from the group consisting of tdiA having the sequence set forth in SEQ ID NO. 16, tdiB having the sequence set forth in SEQ ID NO. 17, tdiC having the sequence set forth in SEQ ID NO. 18, tdiD having the sequence set forth in SEQ ID NO. 19 or tdiE having the sequence set forth in SEQ ID NO. 20. In some preferred embodiments the invention provides a host cell transformed with at least one gene as set forth in SEQ ID NOs. 16-20. In particularly preferred embodiments the host cell is an E coli cell or an Ascomycetes spp. cell.

In yet another preferred embodiment, this invention provides a method of producing terrequinone A comprising steps of: (a) obtaining a fungal cell containing a terrequinone A gene cluster; (b) culturing said fungal cell under conditions sufficient to produce terrequinone A; and (c) isolating said terrequinone A in a substantially purified form. In some preferred embodiments, overexpressing LaeA is accomplished by adding cyclopentanone to the culture medium, transforming the host cell with alc(p) or transforming the host cell with laeA. In other preferred embodiments, step (b) comprises adding L-tryptophan to the culture media or adding of indole 3-pyruvic acid to the culture media. In still other embodiments step (b) comprises expressing the tdiA gene in trans, or expressing the tdiD gene in trans. In still other embodiments the fungal cell is transformed with an isolated terrequinone A gene cluster as set forth in SEQ ID NO. 15.

In another preferred embodiment, the fungal cell is an Aspergillus spp. Cell.

These and other features and advantages of various exemplary embodiments of the articles and methods according to this invention are described in, or are apparent from, the following detailed description of various exemplary embodiments of the compositions and methods according to this invention.

### BRIEF DESCRIPTION OF THE FIGURES

Various exemplary embodiments of the methods of this invention will be described in detail, with reference to the following figures, wherein:

FIGS. 1a and 1b are histograms showing the confirmation of previously identified gene clusters of the LaeA regulon using the genome mining methods described herein. FIG. 1a, represents the sterigmatocystin (ST) gene cluster while FIG. 1b represents the penicillin (PN) gene cluster.

FIGS. 2a and 2b illustrate a transcriptional analysis of the ST gene cluster and genes immediately upstream and downstream of the gene cluster as identified by genomic mining. FIG. 2a Northern analysis and FIG. 2b, schematic explanation of relative locations of the biosynthetic genes in the ST gene cluster. Solid arrows indicate genes in the ST gene cluster and hatched arrows indicate flanking transcripts.

FIGS. 3a and 3b are histograms showing several LaeA-controlled gene clusters identified by genome mining.

FIGS. 4a and 4b are histograms showing another putative secondary metabolite cluster controlled by LaeA. FIG. 4a shows expression ratios ΔlaeA to wild type, FIG. 4b shows expression ratios OE::laeA to wild type, for genes AN0520.2-AN0531.2. Genes putatively belonging to the cluster include (from AN0520.2 (left) to AN0531.2 (right)): a hypothetical protein, a short chain oxidoreductase, a hypothetical protein, a polyketide synthase, a hypothetical protein, a short chain dehydrogenase, a flavonol synthase like protein, three hypothetical proteins (AN0527.2 to AN0529.2), a monooxygenase and a hypothetical protein.

FIGS. 5a and 5b illustrate the tdi gene cluster. FIG. 5a, shows the demarcation of the tdi gene cluster. DNA fragments covering the five tdi cluster genes (probes II and III) were expressed in wild type but not the ΔlaeA mutant. DNA fragments adjacent to the proposed cluster (probes I and IV) were expressed in both wild type and the ΔlaeA mutant. FIG. 5b is agarose gels showing that tdiB is not expressed in a ΔlaeA mutant or a ΔtdiB mutant but is upregulated in the OE:: laeA mutant.

FIG. 6 is a cartoon of the tdi gene cluster illustrating the arrangement of the individual genes, tdiA, tdiB, tdiC, tdiD and tdiE within the cluster; note that ORFs of tdiA and tdiE are found on the antisense strand within the cluster.

FIGS. 7a-7d are data showing the lack of metabolite production in the ΔtdiB mutant. FIG. 7a, is a thin layer chromatography plate having chloroform extracts from wild type and ΔtdiA mutant run a in a hexane:ethyl acetate (4:1) solvent system. The arrow points at the Retention factor R_{f} of the missing metabolite in the ΔtdiB mutant. FIG. 7b, are chromatograms from chloroform extracts from wild type (upper panel) and ΔtdiB-mutant (lower panel) analyzed by HPLC. For wild type, the two major peaks are ST, eluting after 20.9 min, and terrequinone A, eluting after 24.6 min. The chromatograms were recorded at 254 nm, the vertical axis shows milli absorption units (mAU). FIG. 7c, Mass spectroscopy in the positive (upper panel) and negative mode (lower panel). The mass spectrum of the HPLC peak at 24.6 min was extracted, signal intensities are given as relative abundance with the 491.2 signal set as 100%. The peak 491.2 corresponds to the protonated, the 489.2 to the deprotonated molecule. FIG. 7d shows the chemical structure of terrequinone A.

FIG. 8 is a schematic diagram of the terrequinone A molecule illustrating Rotating-frame (nuclear) Overhauser Enhancement (ROE) SpectroscopY (ROESY) analysis correlations with the arrows showing selected Heteronuclear Multiple-Bond Connectivities (HMBC) correlations.

FIG. 9 is a diagram showing the proposed pathway of terrequinone A-biosynthesis. The hypothetical order of the key steps include: deamination (TdiD); adenylation; dimerization (TdiA, Ad=adenylation domain, TE=thioesterase/cyclase domain) and reduction (TdiC). Prenyl transfers might occur separately and at earlier points in the biosynthetic route.

FIGS. 10a and 10b are SDS-PAGE GELS showing expression of Tdi enzymes in E. coli. FIG. 10a: lane 1, standard, ladder; lane 2, TdiA over expression; lane 3, untransformed (UT) E. coli. FIG. 10b: lane 4, TdiD overexpressed in E. coli as for TdiA, lane 2 and purified; lane 5, standard, kD ladder.

### DETAILED DESCRIPTION OF THE EXEMPLARY EMBODIMENTS

### Definitions

Before the present materials methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U. S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N. Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

The term “polyketide synthases” (PKSs) refers to multifunctional enzymes, related to fatty acid synthases (FASs). PKSs catalyze the biosynthesis of polyketides through repeated (decarboxylative) Claisen condensations between acylthioesters, usually acetyl, propionyl, malonyl or methylmalonyl. Following each condensation, they typically introduce structural variability into the product by catalyzing all, part, or none of a reductive cycle comprising a ketoreduction, dehydration, and enoylreduction on the β-keto group of the growing polyketide chain. PKSs incorporate enormous structural diversity into their products, in addition to varying the condensation cycle, by controlling the overall chain length, choice of primer and extender units and, particularly in the case of aromatic polyketides, regiospecific cyclizations of the nascent polyketide chain. After the carbon chain has grown to a length characteristic of each specific product, it is typically released from the synthase by thiolysis or acyltransfer. Thus, PKSs consist of families of enzymes which work together to produce a given polyketide.

Two general classes of PKSs exist. One class, known as Type I PKSs, is represented by the PKSs for macrolides such as erythromycin. These “complex” or “modular” PKSs include assemblies of several large multifunctional proteins carrying, between them, a set of separate active sites for non-iteratively carrying out each step of carbon chain assembly and modification (Cortes et al. (1990) Nature 348: 176; Donadio et al. (1991) Science 252: 675; MacNeil et al. (1992) Gene 115: 119). Structural diversity occurs in this class from variations in the number and type of active sites in the PKSs. This class of PKSs displays a one-to-one correlation between the number and clustering of active sites in the primary sequence of the PKS and the structure of the polyketide backbone. The second class of PKSs, called Type II PKSs, is represented by the synthases for aromatic compounds. Type II PKSs typically have a single set of iteratively used active sites (Bibb et al. (1989) EMBO J. 8: 2727; Sherman et al. (1989 (EMBO J. 8: 2717; Fernandez-Moreno, et al. (1992) J. Biol. Chem. 267:19278).

A “nonribosomal peptide synthase” (NRPS) refers to an enzymatic complex of eukaryotic or prokaryotic origin, that is responsible for the synthesis of peptides by a nonribosomal mechanism, often known as thiotemplate synthesis (Kleinkauf and von Doehren (1987) Ann. Rev. Microbiol, 41: 259-289). Such peptides, which can be up to 20 or more amino acids in length, can have a linear, cyclic (cyclosporin, tyrocidine, mycobacilline, surfactin and others) or branched cyclic structure (polymyxin, bacitracin and others) and often contain amino acids not present in proteins or modified amino acids through methylation or epimerization.

A “module” refers to a set of distinctive polypeptide domains that encode all the enzyme activities necessary for one cycle of polyketide or peptide chain elongation and associated modifications.

A “regulon” refers to genes that are regulated by the same regulatory molecule. The genes of a regulon share a common regulatory element binding site or promoter. The genes comprising a regulon may be located non-contiguously in the genome.

“Isolated” or “purified” or “isolated and purified” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living. As so defined, “isolated nucleic acid” or “isolated polynucleotide” includes nucleic acids integrated into a host cell chromosome at a heterologous site, recombinant fusions of a native fragment to a heterologous sequence, recombinant vectors present as episomes or as integrated into a host cell chromosome. As used herein, the term “substantially purified”, refers to nucleic or amino acid sequences that are removed from their natural environment, isolated or separated, and are at least 60% free, preferably 75% free, and most preferably 90% free from other components with which they are naturally associated. As used herein, an isolated nucleic acid “encodes” a reference polypeptide when at least a portion of the nucleic acid, or its complement, can be directly translated to provide the amino acid sequence of the reference polypeptide, or when the isolated nucleic acid can be used, alone or as part of an expression vector, to express the reference polypeptide in vitro, in a prokaryotic host cell, or in a eukaryotic host cell.

As used herein, the term “exon” refers to a nucleic acid sequence found in genomic DNA that is bioinformatically predicted and/or experimentally confirmed to contribute contiguous sequence to a mature mRNA transcript.

As used herein, the phrase “open reading frame” and the equivalent acronym “ORF” refer to that portion of a transcript-derived nucleic acid that can be translated in its entirety into a sequence of contiguous amino acids. As so defined, an ORF has length, measured in nucleotides, exactly divisible by 3. As so defined, an ORF need not encode the entirety of a natural protein.

The term “microarray” or “array” refers to an ordered arrangement of hybridizable array elements. The array elements are arranged so that there are preferably at least one or more different array elements, more preferably at least 100 array elements, and most preferably at least 1,000 array elements, on a 1 cm² substrate surface. The maximum number of array elements is unlimited, but is at least 100,000 array elements. Furthermore, the hybridization signal from each of the array elements is individually distinguishable. In a preferred embodiment, the array elements comprise polynucleotide representative of fungal-derived polynucleotide sequences.

The term “hybridization complex”, as used herein, refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C₀ t or R₀ t analysis) or between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variations on the traditional peptide linkage joining the amino acids making up the polypeptide. Where the terms are recited herein to refer to a polypeptide, peptide or protein of a naturally occurring protein molecule, the terms are not meant to limit the polypeptide, peptide or protein to the complete, native amino acid sequence associated with the recited protein molecule but shall be understood to include fragments of the complete polypeptide. The term “portion” or “fragment”, as used herein, with regard to a protein or polypeptide (as in “a fragment of the LaeA polypeptide”) refers to segments of that polypeptide which are not naturally occurring as fragments in nature. The segments may range in size from five amino acid residues to the entire amino acid sequence minus one amino acid. Thus, a polypeptide “as set forth in SEQ ID NO: 15 or a fragment thereof” encompasses the full-length amino acid sequence set forth in SEQ ID NO: 15 as well as segments thereof. Fragments of LaeA preferably are biologically active as defined herein.

The terms “nucleic acid” or “oligonucleotide” or “polynucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49:1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U. S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U. S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996) and non-ribose backbones, including those described in U. S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169 176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments. As used herein, oligonucleotide is substantially equivalent to the terms “amplimers”, “primers”, “oligomers”, and “probes”, as commonly defined in the art.

“Nucleic acid sequence” or “nucleotide sequence” or polynucleotide sequence”, as used herein, refers to an oligonucleotide, nucleotide, or polynucleotide, and fragments thereof, and to DNA or RNA of genomic or synthetic origin which may be single- or double-stranded, and represent the sense or antisense strand. Where “nucleic acid sequence” or “nucleotide sequence” or polynucleotide sequence” is recited herein to refer to a particular nucleotide sequence (e.g., the nucleotide sequence set forth in SEQ ID NO:2), “nucleotide sequence”, and like terms, are not meant to limit the nucleotide sequence to the complete nucleotide sequence referenced but shall be understood to include fragments of the complete nucleotide sequence. In this context, the term “fragment” may be used to specifically refer to those nucleic acid sequences which are not naturally occurring as fragments and would not be found in the natural state. Generally, such fragments are equal to or greater than 15 nucleotides in length, and most preferably includes fragments that are at least 60 nucleotides in length. Such fragments find utility as, for example, probes useful in the detection of nucleotide sequences encoding TdiB.

The term “heterologous” as it relates to nucleic acid sequences such as coding sequences and control sequences, denotes sequences that are not normally associated with a region of a recombinant construct, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention. In these instances, the host cell is said to have the nucleic acid in “trans.”

A “coding sequence” or a sequence that “encodes” a particular polypeptide (e.g. a PKS, an NRPS, etc.), is a nucleic acid sequence which is ultimately transcribed and/or translated into that polypeptide in vitro and/or in vivo when placed under the control of appropriate regulatory sequences. In certain embodiments, the boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences. In preferred embodiments, a transcription termination sequence will usually be located 3′ to the coding sequence.

The term “ortholog” refers to genes or proteins which are homologs via speciation, e.g., closely related and assumed to have common descent based on structural and functional considerations. Orthologous proteins function as recognizably the same activity in different species.

Expression “control sequences” refers collectively to promoter sequences, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Recombination” refers to the reassortment of sections of DNA or RNA sequences between two DNA or RNA molecules. “Homologous recombination” occurs between two DNA molecules which hybridize by virtue of homologous or complementary nucleotide sequences present in each DNA molecule.

The terms “stringent conditions” or “hybridization under stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence dependent, and are different under different environmental parameters. An extensive guide to the hybridization of nucleic acids is found in Tijssen (1993) Laboratory Techniques in Biochemistry and Molecular Biology—Hybridization with Nucleic Acid Probes part I chapter 2 Overview of principles of hybridization and the strategy of nucleic acid probe assays, Elsevier, N. Y. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42 C, with the hybridization being carried out overnight. An example of highly stringent wash conditions is 0.15 M NaCl at 72 C for about 15 minutes. An example of stringent wash conditions is a 0.2×SSC wash at 65 C for 15 minutes (see, Sambrook et al. (1989) Molecular Cloning—A Laboratory Manual (2^{nd} ed.) Vol. 1-3 Cold Spring Harbor Laboratory, Cold Spring Harbor Press, NY, for a description of SSC buffer). Often, a high stringency wash is preceded by a low stringency wash to remove background probe signal. An example medium stringency wash for a duplex of, e.g. more than 100 nucleotides, is 1×SSC at 45 C for 15 minutes. An example low stringency wash for a duplex of, e.g. more than 100 nucleotides, is 4-6×SSC at 40 C for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g. when a copy of a nucleic acid is created using the maximum codon degeneracy permitted by the genetic code.

By “genetically engineered host cell” it is meant a host cell where the native PKS and/or NRPS gene cluster has been altered or deleted using recombinant DNA techniques or a host cell into which heterologous PKS and/or NRPS and/or hybrid PKS/NRPS gene cluster has been inserted. Thus, the term would not encompass mutational events occurring in nature. A “host cell” is a cell derived from a prokaryotic microorganism or a eukaryotic cell line cultured as a unicellular entity, which can be, or has been, used as a recipient for recombinant vectors bearing the PKS, NRPS, and/or hybrid gene clusters of the invention. The term includes the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired PKS, are included in the definition, and are covered by the above terms.

“Expression vectors” are defined herein as nucleic acid sequences that direct the transcription of cloned copies of genes/cDNAs and/or the translation of their mRNAs in an appropriate host. Such vectors can be used to express genes or cDNAs in a variety of hosts such as bacteria, bluegreen algae, plant cells, insect cells and animal cells. Expression vectors include, but are not limited to, cloning vectors, modified cloning vectors, specifically designed plasmids or viruses. Specifically designed vectors allow the shuttling of DNA between hosts, such as bacteria-yeast or bacteria-animal cells. An appropriately constructed expression vector preferably contains: an origin of replication for autonomous replication in a host cell, as electable marker, optionally one or more restriction enzyme sites, optionally one or more constitutive or inducible promoters. In preferred embodiments, an expression vector is a replicable DNA construct in which a DNA sequence encoding a one or more PKS and/or NRPS domains and/or modules is operably linked to suitable control sequences capable of effecting the expression of the products of these synthase and/or synthetases in a suitable host. Control sequences include a transcriptional promoter, an optional operator sequence to control transcription and sequences that control the termination of transcription and translation, and so forth.

A “gene cluster” describes DNA fragments or “open reading frames”, or “ORF”, each referring to a nucleic acid open reading frame that encodes a polypeptide or polypeptide domain that has an enzymatic activity used in the biosynthesis of a regulatory domain or secondary metabolite. For example, the tdi gene cluster comprises five ORFs.

A “biological molecule that is a substrate for a polypeptide encoded by a tdi biosynthesis gene” refers to a molecule that is chemically modified by one or more polypeptides encoded by open reading frame(s) of the tdi gene cluster. The “substrate” may be a native molecule that typically participates in the biosynthesis of a terrequinone A, or can be any other molecule that can be similarly acted upon by the polypeptide.

A “polymorphism” is a variation in the DNA sequence of some members of a species. A polymorphism is thus said to be “allelic,” in that, due to the existence of the polymorphism, some members of a species may have the unmutated sequence (i.e. the original “allele”) whereas other members may have a mutated sequence (i.e. the variant or mutant “allele”). In the simplest case, only one mutated sequence may exist, and the polymorphism is said to be diallelic. In the case of diallelic diploid organisms, three genotypes are possible. They can be homozygous for one allele, homozygous for the other allele or heterozygous. In the case of diallelic haploid organisms, they can have one allele or the other, thus only two genotypes are possible. The occurrence of alternative mutations can give rise to trialleleic, etc. polymorphisms. An allele may be referred to by the nucleotide(s) that comprise the mutation.

“Homologous”, as used herein, refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared.times.100. For example, if 6 of 10, of the positions in two sequences are matched or homologous then the two sequences are 60% homologous. By way of example, the DNA sequences ATTGCC and TATGGC share 50% homology. Generally, a comparison is made when two sequences are aligned to give maximum homology.

### The Invention

Previous results from laeA-based genome mining reveals a novel genomic method to identify expressed clusters as described in U. S. Pat. No. 7,053,204 incorporated herein by reference in its entirety for all purposes. This knowledge fills a large gap in existing technology in identifying natural products. Not only does LaeA identify actively synthesized metabolites—even an unknown compound for a given species, as demonstrated in the present case—but it can do so without any structural/chemical or DNA data, requirements of traditional genome mining approaches. LaeA also shows no restrictions in chemical class of metabolite it regulates, be it polyketide, peptide, terpene etc., the single requirement seems to be the arrangement of the biosynthetic genes in a cluster. Furthermore, the demonstration that the A. nidulans OE::laeA allele can function in another species such as A. fumigatus, A. oryzae and A. terreus (Bok, J W; Keller, N P, LaeA, A Regulator of Secondary Metabolism in Aspergillus spp., Eukaryot Cell. (2004) 527-35) and that heterologous gene clusters are regulated by laeA when placed in A. nidulans OE::laeA strains extends the universality of this method to at least all Aspergillus species. This latter point is particularly promising as the potential wealth of Aspergillus bioactive metabolites is enormous. Additionally, examination of several other Ascomycetes indicates a similarity in PKS number to the Aspergilli thus revealing a fungal secondary metabolite capability as rich if not richer than evident from the two published Streptomyces genome projects.

Through the use of laeA based genome mining the inventors have shown that it is possible to identify and isolate novel secondary metabolites, which while the chemical synthesis of such compounds is possible, their synthesis is notoriously difficult and time consuming. In the current instance the inventors describe the production and isolation of a previously unrecognized secondary metabolite of A. nidulans-Terrequinone A.

The present invention provides a novel gene cluster containing five genes (tdiA-E) involved in indole alkaloid synthesis. Disruption of tdiB, encoding an enzyme with prenyltransferase activity, transferring dimethylallylpyrophosphate to C-2 of an indole structure, eliminated the production of the antitumor compound terrequinone A, a metabolite not known from A. nidulans. The invention further provides a method for expressing terrequinone A in a host cell and isolating purified terrequinone A therefrom.

In one preferred embodiment, this invention provides an isolated terrequinone A gene cluster as set forth in SEQ ID NO: 15.

In another preferred embodiment, this invention provides an isolated gene from the terrequinone A gene cluster wherein the gene is selected from the group consisting of tdiA having the sequence set forth in SEQ ID NO. 16, tdiB having the sequence set forth in SEQ ID NO. 17, tdiC having the sequence set forth in SEQ ID NO. 18, tdiD having the sequence set forth in SEQ ID NO. 19 or tdiE having the sequence set forth in SEQ ID NO. 20. In some preferred embodiments the invention provides a host cell transformed with at least one gene as set forth in SEQ ID NOs. 16-20.

In yet another preferred embodiment, this invention provides a method of producing terrequinone A comprising steps of: (a) obtaining a fungal cell containing a terrequinone A gene cluster; (b) culturing said fungal cell under conditions sufficient to produce terrequinone A; and (c) isolating said terrequinone A in a substantially purified form. In some preferred embodiments, overexpressing LaeA is accomplished by adding cyclopentanone to the culture medium, transforming the host cell with alc(p) or transforming the host cell with laeA. In other preferred embodiments, step (b) comprises adding L-tryptophan to the culture media or adding of indole 3-pyruvic acid to the culture media. In still other embodiments step (b) comprises expressing the tdiA gene in trans, or expressing the tdiD gene in trans. In still other embodiments the fungal cell is transformed with an isolated terrequinone A gene cluster as set forth in SEQ ID NO. 15.

In another preferred embodiment, the fungal cell is selected from the group consisting of E. coli, Aspergillus nidulans, Aspergillus fumigatus, Aspergillus oryzae, Aspergillus terreus.

General Experimental Methods

### Fungal Strains and Growth Conditions

Table 1 lists all fungal strains used in this invention. All strains were maintained as glycerol stocks and were grown at 37° C. on glucose minimal medium (GMM), threonine minimal medium (TMM)(Shimizu, K. & Keller, N. P. (2001) Genetics 157, 591-600) or lactose minimal medium (LMM) (Bok, J. W. & Keller, N. P. (2004) Eukaryot. Cell 3, 527-35) supplemented with 30 mM cyclopentanone. Cyclopentanone induces alcA(p) which was used to promote laeA expression in an over expression (OE::laeA) strain. All media contained appropriate supplements to maintain auxotrophs (Adams, T. H. & Timberlake, W. E. (1990) Proc. Natl. Acad. Sci. USA 87, 5405-5409).

TABLE 1
Fungal strains used in this invention

| Fungal Strains | Genotype | Source |
|---|---|---|
| RLMH37 | pyrG89; pyroA4; veA1 | 2 |
| TJW65.7 | pyrG89; pyroA4; ΔtdiC::pyrG; veA1 | 2 |
| rNI773 | pyroA4; veA1 | Jaehyuk Yu |
| RJW40.7 | biA1; methG1; veA1; ΔlaeA::methG | 1 |
| RJW44.2 | biA1; methG1; alcA(p)::laeA::trpC, veA1; ΔlaeA::methG | 1 |
| FGSC26 | biA1; veA1 | FGSC^{a} |

1 Bok, J. W. and Keller, N. P. (2004) LaeA, a regulator of secondary metabolism in Aspergillus spp. Eukaryot. Cell 3, 527-535.
2 Bok J W, et al. (2006) Genomic mining for Aspergillus natural products, Chem Biol. 2006 Jan; 13(1): 31-7.
^{a}FGSC = Fungal Genetics Stock Center

### EXAMPLE 1

### Nucleic Acid Analysis

To identify LaeA regulated genes, first, DNA was obtained. Briefly, DNA extractions from fungal and bacterial strains, restriction enzyme digestion, gel electrophoresis, blotting, hybridization and probe preparation were according to standard methods (Sambrook, J., Fritsch, E. F. and Maniatis, T. (1989). Molecular cloning: a laboratory manual, 2^{nd} edition. (Cold Spring Harbor, N. Y.: Cold Spring Harbor Laboratory Press). Total RNA was isolated from lyophilized mycelia using Trizol reagent (Invitrogen, Carlsbad, Calif.) according to the manufacturers' instructions. RNA blots were hybridized with a 1 kb tdiB PCR product by primers NAIf1 and NAIr1 for the expression of tdiB. The tdi gene cluster boundary was determined by hybridizing a 6 kb PCR product (probe I by primers NCf3 and NCr3), a 4 kb PCR product (probe II by primers NCf4 and NCr4), a 4 kb PCR product (probe III by primers NCf5 and NCr5) and a 3 kb PCR product (probe IV by primers NCf6 and NCr6) to total RNA extracted from wild type and ΔlaeA. Primers are listed in Table 2.

TABLE 2
Primers

| | | | |
|---|---|---|---|
| | | Restriction | SEQ ID |
| Primer | Sequence^{a} | sites | NO. |

| | | | |
|---|---|---|---|
| NcAf1 | 5′AAGAAGCTGAGCCGGGTTGAT | | 1 |
| | G3′ | | |
| Ncar1 | 5′GGCGGAATTCAACCTCGTATC | EcoRI | 2 |
| | CCGTCAGCGT3′ | | |
| NcAf2 | 5′GTAGAAAGCTTCCTCTCACTA | HindIII | 3 |
| | CCCAAGACC3′ | | |
| NcAr2 | 5′CTTGTTCTCAACGCCATCCAG | | 4 |
| | C3′ | | |
| NAIf1 | 5′AGCACTCCTTCCTCCCTCGT | | 5 |
| | G3′ | | |
| NAIr1 | 5′TCCTATACTTGCCACTCAGCC | | 6 |
| | C3′ | | |
| NCf3 | 5′GCGGATTACCAACACTACCAC | | 7 |
| | C3′ | | |
| NCr3 | 5′ACACCGTCTCCGTCTGCCTT | | 8 |
| | C3′ | | |
| NCf4 | 5′AGCCATGACCTGAGTCAGTC | | 9 |
| | AG3′ | | |
| NCr4 | 5′ATGCCGAACTTCCTCTGCGC | | 10 |
| | C3′ | | |
| NCf5 | 5′TTGAGCTTCTTTCTATCAGG | | 11 |
| | GTC3′ | | |
| NCr5 | 5′TTCGACTTTGTCCATTGCCG | | 12 |
| | CC3′ | | |
| NCf6 | 5′TGAATGGCCGGAGAGTGCAC | | 13 |
| | G3′ | | |
| NCr6 | 5′CCTGGTACGTTGCCTTTGTA | | 14 |
| | G3′ | | |

^{a}underlined sequences show placement of restriction sites shown on the right.

### EXAMPLE 2

### Microarray Analysis

Initial analysis of proposed LaeA regulated gene clusters was performed by microarray analysis. Briefly, arrays were generated by Nimblegen, Inc. (Madison, Wis.) for each annotated gene in the A. nidulans genome database (Broad Institute). Each gene was represented by 10 oligonucleotide probe pairs (24 bases each) consisting of a “perfect match” probe identical to a genomic sequence and a “mismatch probe” designed to differ at two positions relative to the perfect match probe. Total RNA of wild type and ΔlaeA strains was prepared in duplicate from FGSC 26 (biA1; veA1) and RJW40.7 (biA1; methG1; ΔlaeA::metG;veA1) grown for 48 hr in GMM using TRIzol® reagent (Invitrogen, Carlsbad, Calif.) followed by RNeasy clean up (Qiagen Inc., Valencia, Calif.), respectively. Total RNA of wild type and OE::laeA strains was prepared in triplicate from FGSC 26 (biA1); veA1) and RJW44.2 (biA1; methG1; alcA(p)::laeA::trpC, veA1; ΔlaeA::methG) grown for 24 hr in LMM with 30 mM cyclopentanone (ICN Biochemicals INC, Aurora, Ohio) after 24 hr in GMM using TRIzol® reagent (Invitrogen, Carlsbad, Calif.) followed by RNeasy clean up (Qiagen Inc., Valencia, Calif.), respectively. Total RNA was spiked with control RNA transcripts, converted to biotinylated cRNA and fragmented following the Affymetrix Expression Analysis Technical Manual.

Hybridization mixtures were prepared according to the array manufacturer's standard protocol using 10 μg biotinylated cRNA and were incubated with the arrays overnight at 45° C. Chips were washed, stained with streptavidin-linked Cy3 dye, and dried according to the manufacturer's protocol. Chips were scanned using a GenePix scanner (Axon Instruments, Union City, Calif.). The data were imported into a Microsoft Access database, mismatch probe signals were subtracted from perfect match signals and averaged across genes. These average signal values were normalized by multiplying every signal value by a scaling factor calculated as 1000 signal units divided by the average signal for the RNA spike controls. For the purpose of calculating ratios, a value of 5 signal units was substituted for genes with negative signal values (where mismatch probe signals exceeded perfect match signals). Genes dependent on LaeA for expression were determined using EBarrays software (Newton, M. A. & Kendziorski C. M. in: The analysis of gene expression data: methods and software. Eds. G. Parmigiani, E. S. Garrett, R. Irizarry & S. L. Zeger (Springer, N. Y.) 2003, pp. 254-271) to identify genes with statistically different signals between mutant and wild type (ΔlaeA mutant strain to wild type or OE::laeA to wild type). Once LaeA-regulated genes had been identified, they were assigned gene identification numbers according to Broad Institute nomenclature for Aspergillus nidulans to verify clustered localization using the publicly accessible genomic sequence (at URL broad.mit.edu/annotation/fungi/aspergillus/). Next, BLAST searches (through Broad Institute) were carried out to check for known homologous genes in other organisms (Nierman, W C et al. Genomic sequence of the pathogenic and allergenic filamentous fungus Aspergillus fumigatus, Nature. 2005 December 22; 438(7071): 1151-6. Erratum in: Nature. 2006 Jan. 26; 439(7075):502).

### EXAMPLE 3

### Identification of LaeA Regulated Gene Clusters

Two of the best characterized fungal secondary metabolite gene clusters are the A. nidulans ST and PN clusters. Prior gene expression data of the A. nidulans ΔlaeA strain compared to wild type showed that selected genes in the ST and PN gene clusters were down regulated in the mutant. To examine the nature and extent of ST and PN gene cluster regulation by LaeA, the inventors analyzed a full genome array for ST and PN gene expression.

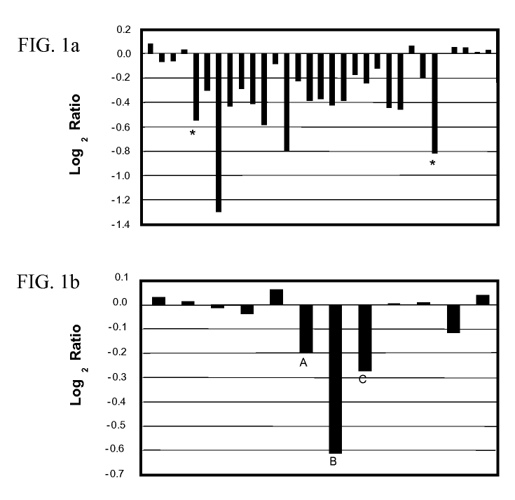

FIGS. 1a and 1b illustrate the log ratios comparing expression of genes (ΔlaeA versus wild type) of the sterigmatocystin (ST) (FIG. 1a) and penicillin (PN) (FIG. 1b) gene clusters. FIG. 1a, represents the sterigmatocystin ST gene cluster. Shown are expression ratios (ΔlaeA to wild type) for genes on Chromosome IV in the region including the ST cluster (AN7800.2-AN7830.2). Asterisks indicate the first (AN7804.2, stcW) and last (AN7825.2, stcA) genes of the cluster, relative to the genome sequence annotation. FIG. 1b, represents the penicillin (PN) gene cluster. Shown are expression ratios (ΔlaeA to wild type) for genes on Chromosome II in the region including the PN cluster (AN2616.2-AN2627.2). PN genes acvA, ipnA, and aatA are indicated (A-C, respectively).

FIGS. 1a and 1b exhibit a pattern the inventors have termed the ‘secondary metabolite cluster signature’ where virtually every gene in the particular cluster is down-regulated in the ΔlaeA strain, in contrast to the undisturbed expression of adjacent genes.

### EXAMPLE 4

### Validation of LaeA Regulated Gene Clusters

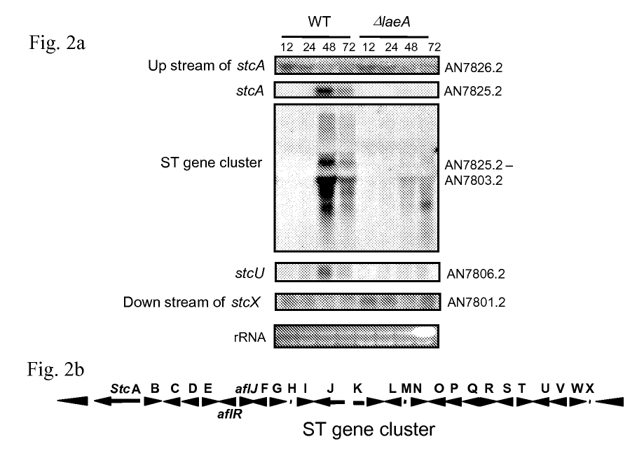

To validate the transcriptional regulation identified above, in Example 3, a transcriptional profile was assessed of the entire 60 kb ST gene cluster by Northern analysis FIG. 2a. Briefly, A. nidulans WT (RDIT2.3) and ΔlaeA (RJW46.4) strains were grown in liquid shaking GMM for 12 h, 24 h, 48 h and 72 h at 37° C., 300 rpm. stcA and stcU are two characterized ST biosynthetic genes near either end of the ST gene cluster (Brown et al., 1996). Blots were hybridized with stcA, stcU, pL11C09 (a cosmid covering AN7825.2-AN7803.2), a stcA flanking PCR product (AN7826.2) and stcX flanking PCR product (AN7801.2). The sequences are found at broad.mit.edu/annotation/fungi/aspergillus/. Ethidium bromide stained rRNA is indicated for loading. FIG. 2b is a schematic explanation of relative locations of biosynthetic genes in ST gene cluster. Solid arrows indicate genes in ST gene cluster, and hatched arrows indicate flanking transcripts (see also, Secondary Metabolic Gene Cluster Silencing in Aspergillus nidulans, Bok, J W et al., Mol Micro, in press). This profile was remarkably similar to that of the array and confirmed that LaeA regulation impacts the cluster region, but not neighboring genes.

### EXAMPLE 5

### Identification of Other LaeA Regulated Gene Clusters

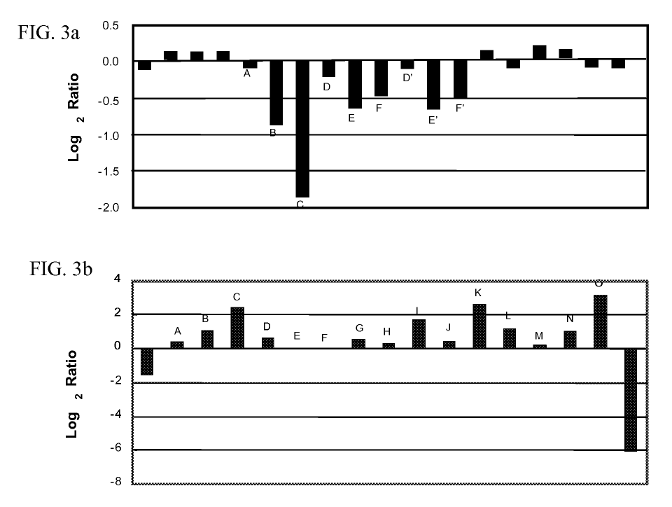

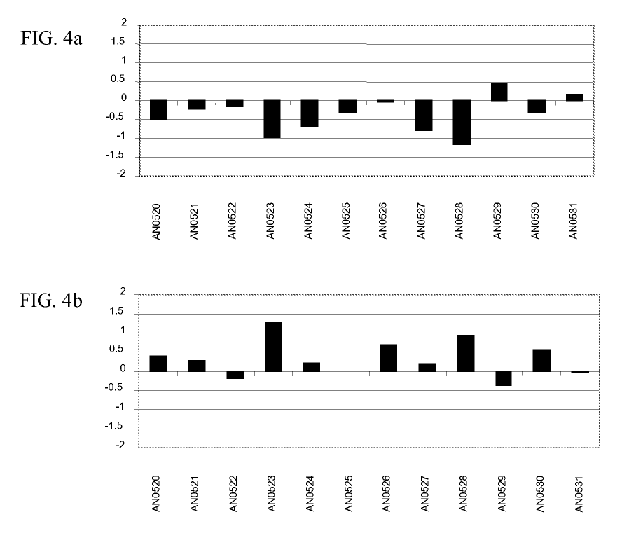

Considering the clear presentation of the secondary metabolism motif for the PN and ST clusters, the inventors then examined the array data for areas of near-contiguous gene suppression in the ΔlaeA strain or near-contiguous gene induction in the OE::laeA strain. Open reading frames found in regions displaying this motif were then examined for the potential to encode secondary metabolite biosynthetic enzymes. Using these criteria, many putative secondary metabolite cluster signatures were identified from both the ΔlaeA and OE::laeA comparisons to wild type. Examples of the effectiveness of this methodology are illustrated by expression ratios (ΔlaeA to wild type) for genes AN8513.2-AN8526.2 (FIG. 3a); AN1588.2-AN1602.2 (FIG. 3b); AN0520.2-AN0531.2 ΔlaeA to wild type (FIG. 4a) and OE::laeA to wild type (FIG. 4b).

FIG. 3a, represents a putative indole alkaloid biosynthetic pathway. Shown are expression ratios (ΔlaeA to wild type) for genes AN8513.2-AN8526.2. Genes belonging to the cluster (confirmed by Northern analysis, see text) putatively encode: an NRPS possessing a peptidyl carrier protein, an adenylating and a thioesterase domain (A), tryptophan dimethylallyltransferase (B), an dehydrogenase/oxidoreductase (C), a homolog of kynurenine aminotransferase (D), and a hypothetical protein (E). Genes C-E are duplicated in the annotated genome sequence (indicated as C′-E′), but this duplication does not exist in the fungal genome, as assessed by PCR. FIG. 3b, represents a putative hybrid secondary metabolite pathway. Shown are expression ratios (OE::laeA to wild type) for genes AN1588.2-AN1602.2. Genes putatively belonging to the cluster include those encoding: hypothetical proteins (A-C), a putative ATPase family protein (D), a polyprenyl synthase (E), a hydroxylmethylglutaryl-coA reductase homolog (e⁻¹²⁰, F), an ent-copalyl diphosphate/ent-kaurene synthase homolog (e⁻¹⁶⁸, G), a translation elongation factor (H), an oxidoreductase (I), a hypothetical protein (J), a P450 monooxygenase (K), a Zn₂-Cys₆ transcription factor (L), a hypothetical protein (M), a cytochrome P450 (N) and a hypothetical protein (O).

The inventors further identified another loci, AN0520-AN0531 regulated by LaeA. FIG. 4a shows expression ratios ΔlaeA to wild type, FIG. 4b shows expression ratios OE::laeA to wild type, for genes AN0520.2-AN0531.2. Genes putatively belonging to the cluster include (from AN0520.2 (left) to AN0531.2 (right)): a hypothetical protein, a short chain oxidoreductase, a hypothetical protein, a polyketide synthase, a hypothetical protein, a short chain dehydrogenase, a flavonol synthase like protein, three hypothetical proteins (AN0527.2 to AN0529.2), a monooxygenase and a hypothetical protein.

### EXAMPLE 6

### Identification of the tdi Gene Cluster

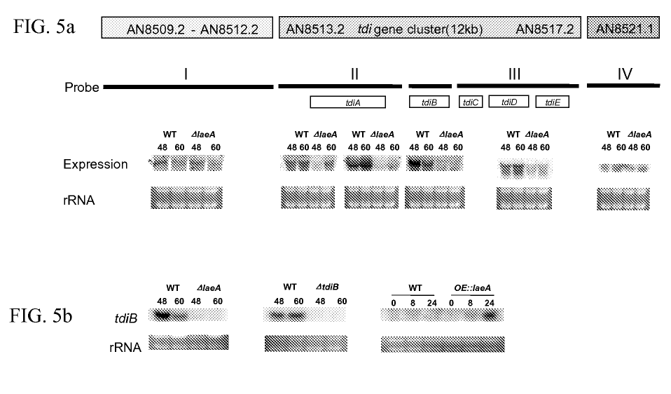

Among the biosynthetic loci identified by the laeA-based genome mining approach for natural product biosynthetic capabilities, one particular locus attracted the inventors' attention. It comprises five open reading frames, transcriptionally regulated by LaeA (FIG. 5a), two of which are similar to genes encoding transferases acting on tryptophan-derived structures (tdiB, dimethylallyl-L-tryptophan synthase, and tdiD, L-kynurenine aminotransferase, respectively), thus being suggestive of indole alkaloid biosynthetic abilities. Other reading frames encode for one hypothetical fungal protein (tdiE), one dehydrogenase/oxidoreductase (tdi, and a monomodular non-ribosomal peptide synthetase (NRPS, tdiA). Interestingly, the deduced TdiA enzyme significantly deviates from the canonical NRPS-architecture (Finking, R. and Marahiel, M. A. (2004), Biosynthesis of nonribosomal peptides. Annu. Rev. Microbiol. 58, 453-488) as it merely comprises an adenylation domain and a peptidyl carrier domain, but lacks a condensation domain to form a peptide bond. However, it includes—typically a bacterial NRPS feature—a thioesterase-domain that releases the completed peptide from the enzyme (Doekel, S, and Marahiel, M. A. (2001). Biosynthesis of natural products on modular peptide synthetases. Metabol. Eng. 3, 64-77). The highest similarity across the whole deduced amino acid sequence was found to putative NRPSs of bacterial origin (Ralstonia solanacearum and Burkholderia pseudomallei, ENTREZ accession numbers NP_{—}522978 and YP_{—}110151, respectively). These findings suggest either an enzyme acting in trans on a second condensing NRPS encoded outside the cluster, a solely adenylating (i.e activating) enzyme, or a non-functional pseudogene perhaps as a remnant of an ancient horizontal gene transfer.

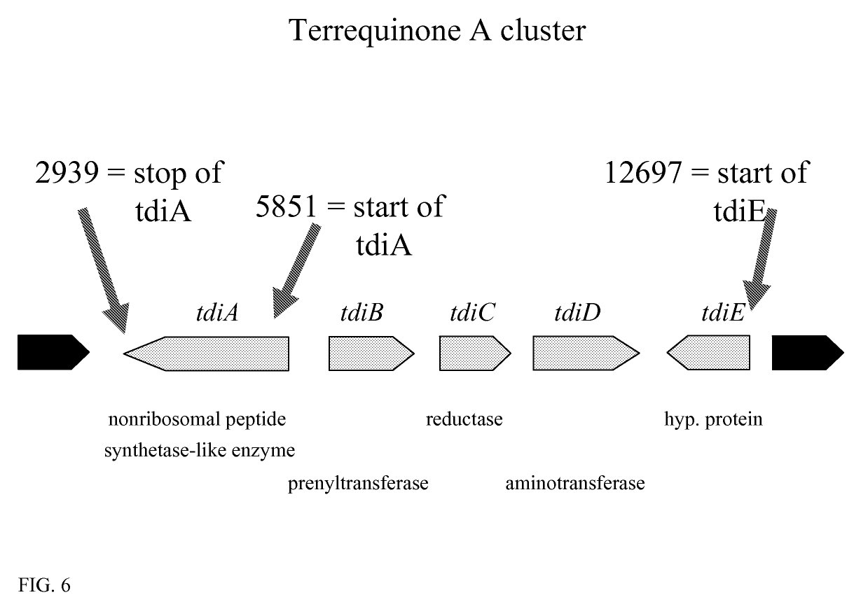

Further analysis by the inventors of the tdi gene cluster revealed that the ORFs for tdiA and tdiE are carried on the antisense strand as shown in FIG. 6. SEQ ID NO: 15 comprises the entire tdi gene cluster beginning at the stop codon for tdiA and ending at the start codon ‘CAT’ of tdie as read 5′-3′ from the sense strand of the genomic single stranded sequence. It is important to note that while the tdi gene cluster is entered in the Aspergillus database maintained at the Broad Institute, (broad.mit.edu/annotation/fungi/aspergillus/) erroneously genes AN8515.2 through AN8517.2 have been duplicated as AN 8518.2 through AN8520.2. However, to the inventors knowledge, the nucleotide sequence given in SEQ ID NO: 15 is the entire tdi gene cluster from A. nidulans. Further, it should be appreciated that regulatory sequences for the tdi cluster can be found downstream of tdiA (e.g., 2938 and lower) and upstream of tdiE (e.g. 12,698 and higher) and contain the native regulatory sequence for tdi expression. These sequences are publicly available from the Broad Institute's A. nidulans database.

### EXAMPLE 7

### Disruption of tdiB Gene

To determine if the identified tdi gene cluster produced a bona fide secondary metabolite, the inventors inactivated the second gene in the cluster, tdiB. tdiB shows similarity to Claviceps fusiformis dmaW, a gene encoding dimethylallyl-L-tryptophan synthase (Wang, J., Machado, C. and Schardl, C. L. (2004), The determinant step in ergot alkaloid biosynthesis by a grass endophyte, Fungal Genet. Biol. 41, 189-198), and other fungal L-tryptophan dimethylallyltransferases, catalyzing the first committed step in the ergot alkaloid pathway. Briefly, PCR techniques were applied to create a tdiA disruption cassette where the tdiB open reading frame was replaced with the A. parasiticus pyrG selection marker. The disruption cassette was constructed by ligating a 1.1 kb DNA fragment upstream of the tdiB start codon (primers NcAf1 and NcAr1, the latter with an EcoRI site) and a 1.1 kb DNA fragment downstream of the tdiB stop codon (primers ncAf2 and NcAr2, the latter with a HindIII site) to the EcoRI and HindIII side of the A. parasiticus pyrG marker gene, respectively, obtained from pBZ5 (Skory, C. D., Chang, P. K., Cary, J. and Linz, J. E. (1992), Isolation and characterization of a gene from Aspergillus parasiticus associated with the conversion of versicolorin A to sterigmatocystin in aflatoxin biosynthesis. Appl. Environ. Microbiol. 58, 3527-3537). Three μl of the ligation mixture was used to amplify the resultant 5 kb disruption cassette by Triple master PCR kit (Eppendorf, Westbury, N. Y.). Twenty μl of the PCR product was purified by G-50 column (Pharmacia) and then used for the disruption of the tdiB gene. Primers are listed in Table 2. PfuUltra (Stratagene) was used for PCR reactions of 5′ and 3′ flanking region of the cassette. Strain RLMH37 was transformed by the PCR fragment. Fungal transformation essentially followed that of Shimizu and Keller (Shimizu, K. and Keller, N. P. (2001), Genetic involvement of a cAMP-dependent protein kinase in a G protein signaling pathway regulating morphological and chemical transitions in Aspergillus nidulans, Genetics 157, 591-600) with the modification of embedding the protoplasts in top agar (0.75%) rather than spreading them by a glass rod on solid media. Five out of 27 transformants were confirmed by Southern hybridization to contain a tdiB gene replacement. One of disruptants, TJW65.7 was used for subsequent experiments.

### EXAMPLE 8

### Chemical Analysis of the ΔtdiB-Mutant

The A. nidulans TJW65.7 and A. nidulans wild type were grown in 100 ml liquid GMM. The cultures were fermented at 37° C. and 200 rpm, for three days. Upon harvest, the fermentation broth was centrifuged (10 min, 2,700 g). The mycelia were extracted with 30 ml chloroform. The supernatant was extracted separately with an equal volume of chloroform. The organic layers were evaporated in vacuo, then redissolved in 300 μl methanol and subjected to High Performance Liquid Chromatography (HPLC). Because no differences were found, both extracts were pooled (for preparative purposes, a 4 liter fermentation was used, the solvent volumes scaled up accordingly, and the broth extracted twice). Analytical HPLC was performed on a Waters liquid chromatograph with an Xterra MS C-18 column (100×4.6 mm), and a C-18 guard column, maintained at 35° C.: detection at 254 nm (diode array acquisition: 220-500 nm). Solvent A: 0.5% (v/v) acetic acid in H₂O, solvent B: 0.5% (v/v) acetic acid in acetonitrile, flow rate: 0.5 ml min⁻¹. The gradient was: initial hold for 3 min at 20% B, then within 23 min to 95% B. Liquid chromatography-Mass Spectroscopy (LC/MS) in analytical and preparative scale was performed on an Agilent 1100 integrated system equipped with a Zorbax Eclipse XDB C-8 column (150×4.6 mm, 5 μm particle size), and C-8 guard column, essentially applying the conditions described for HPLC, using atmospheric pressure chemical ionization (APCI), and switching between positive and negative mode. For preparative HPLC a Zorbax SB C-18 column (150×9.4 mm) and a C-18 guard column were used; flow rate was 3.5 ml min⁻¹. Thin layer chromatography was carried out on silica gel 60 plates with hexane:ethyl acetate (4:1, v/v) as mobile phase. For NMR experiments, the pure compound was dissolved in acetone-d₆.

### EXAMPLE 9

### Characterization of the ΔtdiB-Mutant

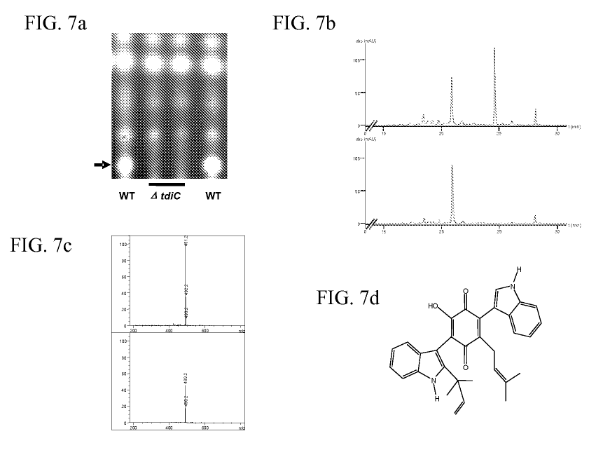

Similar to other secondary metabolite genes (Bok, J. W. and Keller, N. P. (2004), LaeA, a regulator of secondary metabolism in Aspergillus spp. Eukaryot. Cell 3, 527-535), tdiB is not only repressed in the ΔlaeA mutant but also upregulated in a laeA over expression background (FIG. 5b). Disruption of tdiB resulted in a mutant (TJW65.7) unable to produce a compound that appears yellow-orange on TLC under UV light (FIG. 7a). HPLC-UV/Vis and LCAMS analyses with extracts of the TJW65.7 mutant and wild type identified ST as a known compound from A. nidulans in both samples (FIG. 7b). However, the ΔtdiB sample was lacking a second major substance (FIG. 7c). This compound was purified from the wild type and assigned a molecular mass of m/z=490 (m/z=489 [M-H⁺] and 491 [M+H⁺]). Full one- and two dimensional NMR analyses (see Table 3) identified the compound as the recently identified terrequinone A (FIG. 7d), a fungal bisindolyl-quinone with inhibitory properties on tumor cell lines (He, J., et al., (2004), Cytotoxic and other metabolites of Aspergillus inhabiting the rhizosphere of Sonoran desert plants. J. Nat. Prod. 67, 1985-1991) which was not known to be produced by Aspergillus nidulans. The data shown in Table 3 provide the NMR spectra for terrequinone A. The spectra were recorded on a Bruker Avance spectrometer where: ¹H-NMR (500 MHz, acetone-d₆, acetone-d₅, =2.04 ppm), ¹³C-NMR (125.7, acetone-d₆).

TABLE 3*

| | | ¹H | | ¹³C | |
|---|---|---|---|---|---|
| Position # | δ [ppm] | m | J [Hz] | δ [ppm] | m |
| 1 | | | | 184.4 | s |
| 2 | | | | 154.0 | s |
| 3 | | | | 117.9 | s |
| 4 | | | | 188.2 | s |
| 5 | | | | 147.0 | s |
| 6 | | | | 135.1 | s |
| 7a | 3.25 | dd | 13, 7 | 28.7 | t |
| 7b | 3.35 | dd | 13, 7 | | |
| 8 | 5.06 | brdd | 6, 6 | 122.5 | d |
| 9 | | | | 133.3 | s |
| 10 | 1.28 | brs | | 17.8 | q |
| 11 | 1.55 | brs | | 25.8 | q |
| 1′ | | | | 102.8 | s |
| 2′ | | | | 143.3 | s |
| 3′-NH | 10.05 | s | | | |
| 4′ | | | | 137.2 | s |
| 5′ | | | | 129.9 | s |
| 6′ | 7.22 | d | 8 | 119.7 | d |
| 7′ | 6.92 | ddd | 8, 7, 1 | 119.5 | d |
| 8′ | 7.03 | ddd | 8, 7, 1 | 121.7 | d |
| 9′ | 7.32 | d | 8 | 111.4 | d |
| 10′ | | | | 39.9 | d |
| 11′ | 6.17 | dd | 18, 10.5 | 146.8 | d |
| 12′a | 5.00 | dd | 10.5, 1 | 111.5 | t |
| 12′b | 5.10 | dd | 17.5, 1 | | |
| 13′ | 1.52 | s | | 27.2 | q |
| 14′ | 1.52 | s | | 27.8 | q |
| 1″ | | | | 108.2 | s |
| 2″ | 7.47 | brs | | 127.5 | d |
| 3″-NH | 10.73 | s | | | |
| 4″ | | | | 136.4 | s |
| 5″ | | | | 128.0 | s |
| 6″ | 7.39 | d | 8 | 120.7 | d |
| 7″ | 7.09 | ddd | 8, 7, 1 | 120.5 | d |
| 8″ | 7.18 | ddd | 8, 7, 1 | 122.5 | d |
| 9″ | 7.51 | d | 8 | 112.5 | d |

*NMR spectra were recorded on a Bruker Avance spectrometer: ¹H-NMR (500 MHz, acetone-d₆, acetone-d₅ = 2.04 ppm), ¹³C-NMR (125.7, acetone-d₆).

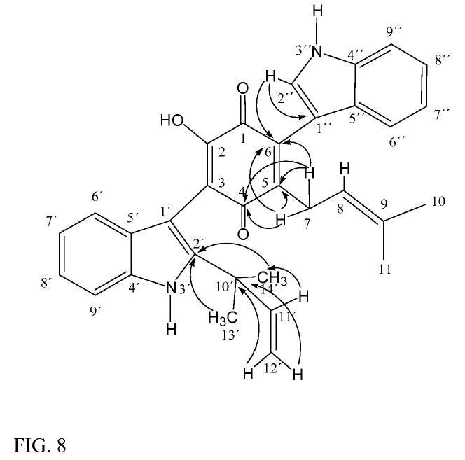

An analysis of the molecule identified in Example 9 using Rotating-frame (nuclear) Overhauser Enhancement (ROE) SpectroscopY (ROESY) was performed. FIG. 8 is a molecular model of the terrequinone A molecule with the arcs representing selected ROESY correlations and the arrows representing selected Heteronuclear Multiple-Bond Connectivities (HMBC) correlations derived from the NMR analysis.

### EXAMPLE 10

### Terreqinone Biosynthesis

Although feeding experiments have led to proposed biosynthetic routes for this class of compounds (Arai, K. and Yamamoto, Y. (1990), Metabolic products of Aspergillus terreus. X. Biosynthesis of Asterrequinones, Chem. Pharm. Bull. 38, 2929-2932), gene clusters have not been identified for these metabolites. Matching the chemical structure of terrequinone A to the tdi-cluster explains the lack of condensation domain within the TdiA enzyme, as no amide bond has to be closed, and implicates a speculative, yet plausible order for the key biosynthetic events: i) deamination of L-tryptophan to indolepyruvic acid by the transaminase TdiD; ii) activation to AMP-indolepyruvic acid by TdiA (adenylation domain) whose nonribosomal code points to an arylic acid rather than amino acid activating function (May, J. J., Kessler, N., Marahiel, M. A. and Stubbs, M. T. (2002). Crystal structure of DhbE, an archetype for aryl acid activating domains of modular nonribosomal peptide synthetases. Proc. Natl. Acad. Sci. USA 99, 12120-12125); iii) dimerization of two activated indolepyruvic acid monomers to the core quinone structure which might be accomplished by the TdiA thioesterase domain, analogous to the cyclization activity of the tyrocidine thioesterase domain (Trauger, J. W., Kohli, R. M., Mootz, H. D., Marahiel, M. A. and Walsh, C. T. (2000), Peptide cyclization catalysed by the thioesterase domain of tyrocidine synthetase. Nature 407, 215-218); and finally iv) the oxidoreductase TdiC might play a role in reducing the keto groups of the quinone core perhaps to prepare it for the prenyl transfer. However, the full metabolic pathway (e.g. at which time the two tailoring prenyl transfer reactions occur) remains elusive and will be the subject of further genetic and biochemical investigations.

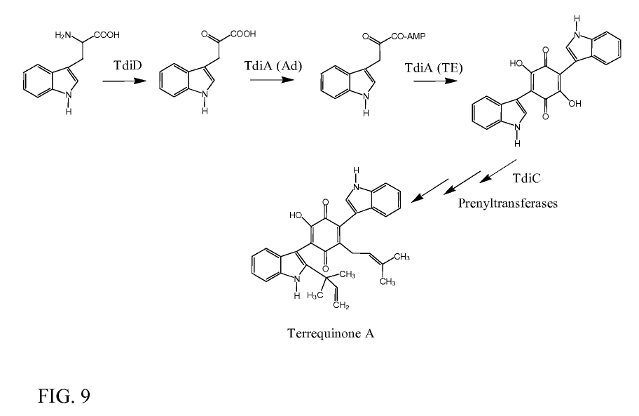

Without being held to any particular theory as to the specific biosynthetic steps, FIG. 9 represents a hypothetical biosynthetic pathway for terrequinone A in Aspergillus wherein TdiA adenylates and dimerizes indole pyruvic acid to the terrequinone core structure; TdiB is a prenyltransferase, transferring dimethylallylpyrophosphate to C-2 of an indole structure; TdiC is an NADPH-dependent reductase, reducing a keto group to an alcohol; TdiD is a transaminase, removing the NH₂ from tryuptophan and transfers NH₂ to a 2-oxo-carboxylic acid and may supply TdiA with the substrate. The function of TdiE has not yet been determined however, its presence is necessary for terrequinone production.

It should be appreciated that, while terrequinone A expression can be induced by the addition of cyclopentanone to the growth medium thereby inducing alcA(p) and promoting laeA expression, terrequinone A production can be induced by other means. For example, the growth media can be supplemented with L-tryptophan or indole 3-pyruvic acid the precursors in Terrequinone A biosynthesis. In addition, by introducing extra copies, in trans, of the entire tdi gene cluster or the first gene, tdiD in the biosynthetic pathway into Aspergilli, the system may be induced to produce terrequinone A.

### EXAMPLE 11

### Expression and Confirmation of Individual tdi Genes

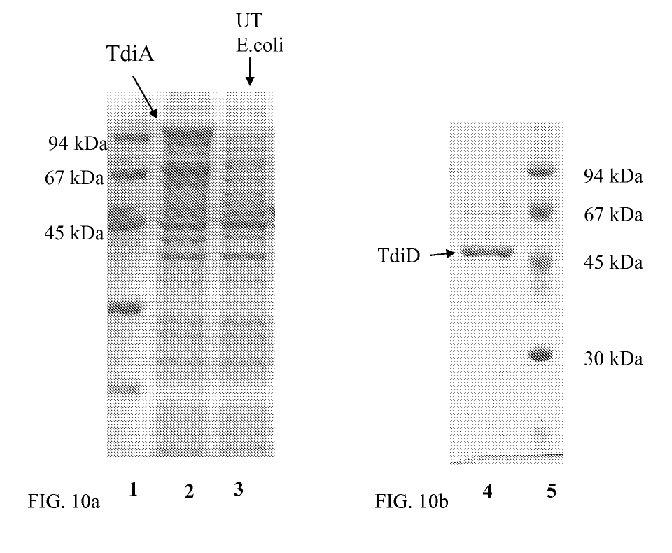

Upon identification of the tdi gene cluster, the individual tdi genes were cloned and cDNA was synthesized for each ORF where tdiA is SEQ ID NO 16, tdiB is SEQ ID NO: 17, tdiC is SEQ ID NO 18, tdiD is SEQ ID NO: 19 and tdiE is SEQ ID NO: 20. As discussed above the gene products have postulated functions where, TdiA adenylates and dimerizes indole pyruvic acid to the terrequinone core structure; TdiB is a prenyltransferase, transferring dimethylallylpyrophosphate to C-2 of an indole structure; TdiC is an NADPH-dependent reductase, reducing a keto group to an alcohol; TdiD is a transaminase, removing the NH₂ from tryptophan and transfers NH₂ to a 2-oxo-carboxylic acid and may supply TdiA with substrate. Expression and purification of the genes of the tdi gene cluster was performed as follows: cDNA of tdiA and tdiD was obtained form total A. nidulans RNA and first strand synthesis was performed with Promega (Madison, Wis.) IMProm Reverse transcriptase. Second strand synthesis was accomplished using Promega Pfu DNA polymerase. The products were ligated to PET28b, cut with Nde1/EcoR1. E. coli BL21 (DE3) was transformed with the resulting constructs using electroporation. Expression was induced with IPTG at a final concentration of 1 mM for 4 h. Cells were lysed and total proteins were purified on a Nickel-agarose column (Qiagen, Valencia, Calif.). FIGS. 10a and 10b show the overexpression of TdiA and TdiD. FIG. 10a represents the unpurified TdiA protein (lane 2) with lane 3 showing an untransformed control (lane 1 is a standard kD ladder). FIG. 10b shows expression of the TdiD protein expressed as for TdiA and purified by nickel agarose column.

As disclosed herein, the inventors have described a hitherto unidentified Aspergillus secondary metabolite. The metabolite, terrequinone A is produced in response to an overexpression of the secondary metabolite regulatory polypeptide LaeA and has been shown to have antitumor properties. As described, according to the invention the biosynthesis of terrequinone can be induced by providing precursors in the terrequinone biosynthetic pathway in the culture media. Alternatively, genes encoding the tdi cluster or the initial genes in the cluster can be put into expression vectors and transformed into host cells so as to drive the production of terrequinone A.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

What is claimed is:

1. An isolated terrequinone A gene cluster as set forth in SEQ ID NO: 15.

2. A host cell transformed with an isolated terrequinone A gene cluster as set forth in SEQ ID NO: 15.

3. The host cell according to claim 2 wherein the gene cluster is operatively linked to an inducible promoter.

4. The host cell of claim 2, wherein the host cell is a bacteria, a fungus, or a yeast cell.

5. The host cell of claim 4, wherein the bacteria is E. coli.

6. The host cell of claim 5, wherein the fungus is an Ascomycetes.

7. An isolated gene from the terrequinone A gene cluster wherein the gene is selected from the group consisting of tdiA having the sequence set forth in SEQ ID NO. 16, tdiB having the sequence set forth in SEQ ID NO. 17, tdiC having the sequence set forth in SEQ ID NO. 18, tdiD having the sequence set forth in SEQ ID NO. 19 or tdiE having the sequence set forth in SEQ ID NO. 20.

8. A host cell transformed with at least one isolated gene according to claim 7.

9. The host cell of claim 8, wherein the host cell is a bacterial cell.

10. The host cell of claim 9 wherein the bacterial cell is an E. coli cell.

11. The host cell of claim 8, wherein the host cell is a fungal cell.

12. The host cell of claim 11, wherein the fungal cell is an Ascomycete spp.

13. A method of producing terrequinone A comprising steps of:
(a) obtaining a fungal cell containing a terrequinone A gene cluster;
(b) culturing said fungal cell under conditions sufficient to produce terrequinone A; and
(c) isolating said terrequinone A in a substantially purified form.

14. The method of claim 13, wherein step (b) comprises overexpressing LaeA.

15. The method of claim 14, wherein overexpressing LaeA is accomplished by adding cyclopentanone to the culture medium, transforming the host cell with alc(p) or transforming the host cell with laeA.

16. The method of claim 13, wherein step (b) comprises adding L-tryptophan to the culture media or adding of indole 3-pyruvic acid to the culture media.

17. The method of claim 13, wherein step (b) comprises expressing the tdiA gene in trans, or expressing the tdiD gene in trans.

18. The method of claim 13, wherein the fungal cell is transformed with an isolated terrequinone A gene cluster as set forth in SEQ ID NO. 15.

19. The method of claim 13, wherein the fungal cell is an Aspergillus spp.
